(19) United States
(12) Patent Application Publication
(10) Pub. No.: US 2025/0222145 A1
(43) Pub. Date: Jul. 10, 2025

(54) TRACER COMPOSITION FOR RADIOACTIVE PET DIAGNOSIS, INTERMEDIATE THEREOF, AND PRODUCTION METHOD THEREOF

(71) Applicant: OSAKA UNIVERSITY, Osaka (JP)

(72) Inventors: Sadahiro NAKA, Osaka (JP); Tadashi WATABE, Osaka (JP); Mitsunori KIRIHATA, Osaka (JP); Yoichiro OHTA, Osaka (JP)

(73) Assignee: OSAKA UNIVERSITY, Osaka (JP)

(21) Appl. No.: 18/699,615

(22) PCT Filed: Oct. 3, 2022
(86) PCT No.: PCT/JP2022/037004

§ 371 Date: Apr. 9, 2024

(30) Foreign Application Priority Data
Oct. 11, 2021 (JP) 2021-166806

(51) Int. Cl. A61K 51/04; C07C 229/36; C07F 5/02

(52) U.S. Cl. CPC A61K 51/0497; A61K 51/0402; C07C 229/36; C07F 5/025; A61K 2121/00; A61K 2123/00

(57) ABSTRACT

Provided are a novel tracer drug for radioactive PET diagnosis having high accumulation properties and retention properties at a tumor site and an efficient production method of the tracer drug (label synthesis method).

The present invention provides a novel tracer compound having improved accumulation and retention properties at a tumor site while maintaining high selectivity to an LAT1 by introducing a methoxy (OMe) group into the 4-position of a benzene ring of an α-methyltyrosine (AMT) skeleton. The present invention also provides a production method capable of efficiently synthesizing the novel tracer compound in high yield by a method of label synthesis using ¹⁸F-hydrogen fluoride (¹⁸F—HF) by using a novel labeled intermediate (precursor) obtained by introducing a boronic acid ester into the 3-position of the benzene ring of the AMT skeleton.

### TECHNICAL FIELD

The present invention relates to a tracer composition for radioactive PET diagnosis targeting an LAT1, an intermediate thereof, and a production method thereof. The present invention further relates to a kit for use in synthesis of the tracer composition for radioactive PET diagnosis.

### BACKGROUND ART

Among amino acid transporters that are membrane proteins required for intracellular uptake of neutral branched chain amino acids containing many essential amino acids and aromatic amino acids, a large neutral amino acid transporter called an L-type amino acid transporter 1 (LAT1) is known to act as an amino acid uptake port of cancer cells. Although the LAT1 is not present in normal cells in most tissues, the LAT1 is specifically expressed in cancer cells and is responsible for supplying amino acids as nutrients in cancer tissues (Non-Patent Literature 1).

Therefore, an effort has been made to develop a tracer drug (probe for PET) targeting the LAT1 in order to use the tracer drug in diagnosing cancer by radioactive positron emission tomography (PET). However, a conventional tracer drug has a problem in that the drug does not have high selectivity to the LAT1 or has insufficient selectivity to tumor.

In contrast, a study has been reported in which fluoro-α-methyltyrosine (¹⁸F-FAMT), which is a compound obtained by labeling, using ¹⁸F, α-methyltyrosine (AMT), which is known as an amino acid incorporated into the LAT1 specific for cancer cells more than an amino acid transporter of normal cells, is used as a tracer drug for radioactive PET diagnosis (Non-Patent Literature 2). However, it has been confirmed that the ¹⁸F-FAMT exhibits high selectivity to the LAT1 but disappears from a tumor site at an early stage. Accordingly, at present, practical application of the ¹⁸F-FAMT is still insufficient. Therefore, it has been desired to develop a novel tracer drug having higher accumulation properties and retention properties at a tumor site.

In addition, in the production of the conventional ¹⁸F-FAMT, in order to directly introduce a fluorine atom (¹⁸F) into a benzene ring, label synthesis using ¹⁸F—F₂ gas (¹⁸F⁺ method) has been performed. However, in such a method, ¹⁸F—F₂ obtained by deuteron irradiation using neon gas as a target has a problem that the obtained radioactivity is small and, as a result, an amount of the ¹⁸F-FAMT that can be produced at one time is limited to a very small amount (typically, PET inspection for about one person and two people). So far, although label synthesis (¹⁸F⁻ method) using ¹⁸F-hydrogen fluoride (¹⁸F—HF) has been established for an existing tracer drug such as ¹⁸F-fluoro-L-dopa, a report has not been made about an ¹⁸F-FAMT-based compound in the current situation.

### CITATION LIST

### Non-Patent Literature

Non-Patent Literature 1: Kanai et al., J. Biol. Chem. 273: 23629, 1998
Non-Patent Literature 2: Kaira et al., Clin Cancer Res. 13: 6369-6378, 2007

### SUMMARY OF INVENTION

### Technical Problem

From such a background, an object of the present invention is to provide a novel tracer drug for radioactive PET diagnosis having high accumulation properties and retention properties at a tumor site. At the same time, it is also an object to provide an efficient production method of the tracer drug (label synthesis method).

### Solution to Problem

As a result of intensive studies to solve the above-described problems, the present inventors have found that by introducing a methoxy (OMe) group in place of a hydroxyl (OH) group into the 4-position of a benzene ring of an α-methyltyrosine (AMT) skeleton, it is possible to provide a novel tracer compound having improved accumulation properties and retention properties at a tumor site while maintaining high selectivity to the LAT1. This is based on the idea that, in the conventional α-methyltyrosine derivative such as ¹⁸F-FAMT, a hydroxyl group (OH group) at the 4-position of a benzene ring is easily incorporated into the renal tubules by interaction with an organic anion transporter (OAT1), and incorporation into the kidney is reduced by introducing an appropriate substituent for suppressing the interaction instead of the OH group, thereby improving the accumulation properties and retention properties of the ¹⁸F-labeled tracer compound in the tumor.

Furthermore, the present inventors have also found that by using a novel labeled intermediate (precursor) obtained by introducing a boronic acid ester into the 3-position of the benzene ring of the AMT skeleton, the novel tracer compound can be efficiently synthesized in high yield by a method of label synthesis (¹⁸F⁻ method) using ¹⁸F-hydrogen fluoride (¹⁸F—HF) instead of conventional label synthesis (¹⁸F⁺ method) using ¹⁸F—F₂ gas. Based on these findings, the present invention has been completed.

That is, the present invention, in one aspect, relates to a composition for radioactive PET diagnosis including a novel tracer compound of an LAT1 targeting type, and more particularly provides:
<1> A tracer composition for radioactive PET diagnosis, the tracer composition targeting an L-type amino acid transporter 1 (LAT1) and including a compound represented by a following formula (I) or a pharmaceutically acceptable salt thereof:

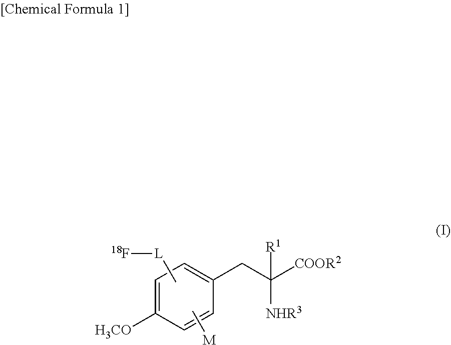

(in the formula, L is a direct bond or an optionally substituted C₁ to C₅ alkylene group; M is a hydrogen atom or a halogen atom; R¹ is the hydrogen atom or an optionally substituted C₁ to C₅ alkyl group; R² is the hydrogen atom, the C₁ to C₅ alkyl group, a C₆ to C₁₄ aryl group, or a C₇ to C₁₆ arylalkyl group; and R³ is the hydrogen atom or a C₁ to C₃ alkyl group.);
<2> The composition according to <1>, in which L is the direct bond; and M is the hydrogen atom;
<3> The composition according to <1>, in which the compound is represented by a following formula (II):

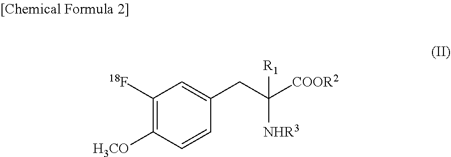

(In the formula, definitions of R¹, R², and R³ are the same as definitions of the formula (I), respectively);
<4> The composition according to any one of <1> to <3>, in which R¹ is the optionally substituted C₁ to C₅ alkyl group; R² is the hydrogen atom or the C₁ to C₅ alkyl group; and R³ is the hydrogen atom;
<5> The tracer composition according to any one of <1> to <4>, being used for a diagnosis of cancer;
<6> The composition according to any one of <1> to <5>, further including a pharmaceutical compound for cancer treatment;
<7> The composition according to any one of <1> to <5>, being used in combination with a pharmaceutical compound for cancer treatment; and
<8> The composition according to <6> or <7>, in which the pharmaceutical compound for cancer treatment is a compound containing astatin-211 (²¹¹At) in a molecule.

In another aspect, the present invention also relates to a precursor compound for synthesizing a compound represented by the formula (I), and provides
<9> An intermediate compound represented by the following formula (A) or a pharmaceutically acceptable salt thereof:

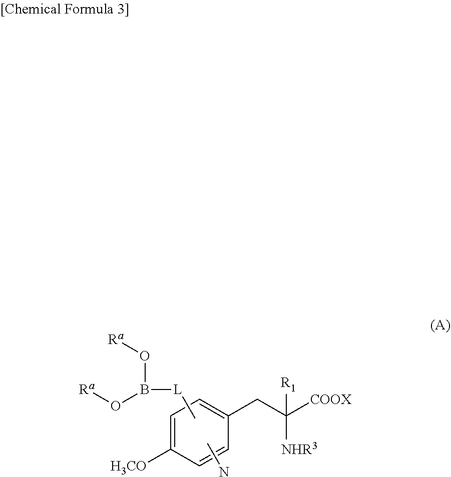

(in the formula, L is a direct bond or an optionally substituted C₁ to C₅ alkylene group; M is a hydrogen atom or a halogen atom; respective pieces of R^{a} may be identical or may be different from each other, in which each of the pieces of R^{a} independently represents the hydrogen atom or an optionally substituted C₁ to C₅ alkyl group, and when all the pieces of R^{a} are the C₁ to C₅ alkyl group, the two pieces of R^{a} may form a cyclic ester structure together with an O atom to which the two pieces of R^{a} are linked; R¹ is the hydrogen atom or the optionally substituted C₁ to C₅ alkyl group; and X and Y are respectively protective groups which may be identical or may be different from each other.); and
<10> The intermediate compound according to <9>, in which X is a group selected from a group formed of a tert-butyl group, a benzyl group, an acetyl group, a benzoyl group, and an alkyl group; and Y is a group selected from a group formed of a tert-butoxycarbonyl group, a benzyloxycarbonyl group, a 9-fluorenylmethyloxycarbonyl group, an allyloxycarbonyl group, a 2,2,2-trichloroethoxycarbonyl group, and a 2-(trimethylsilyl)ethoxycarbonyl group.

In a further aspect, the present invention also relates to a production method of a compound represented by a formula (I), and provides:
<11> A production method of a compound represented by a following formula (I):

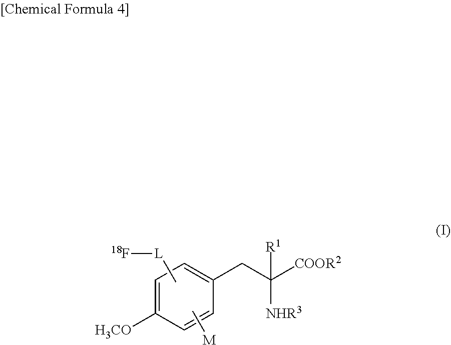

(in the formula, L is a direct bond or an optionally substituted C₁ to C₅ alkylene group; M is a hydrogen atom or a halogen atom; R¹ is the hydrogen atom or an optionally substituted C₁ to C₅ alkyl group; R² is the hydrogen atom, the C₁ to C₅ alkyl group, a C₆ to C₁₄ aryl group, or a C₇ to C₁₆ arylalkyl group; and R³ is the hydrogen atom or a C₁ to C₃ alkyl group.), the production method including a step i) and a step ii), in which:
the step i) is a step of adding hydrogen fluoride (H¹⁸F) to a compound represented by a formula (A) in presence of a catalyst so as to obtain a compound represented by a following formula (B)

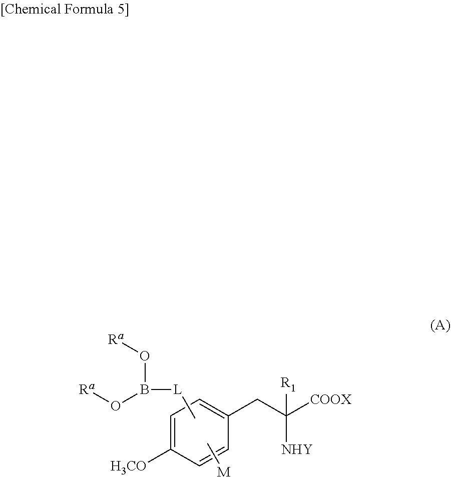

(in the formula, definitions of L, M, and R¹ are the same as definitions of the formula (I), respectively; respective pieces of R^{a} may be identical or different from each other, in which each of the pieces of R^{a} independently represents the hydrogen atom or the optionally substituted C₁ to C₅ alkyl group, and when all the pieces of R^{a} are the C₁ to C₅ alkyl group, the two pieces of R^{a} may form a cyclic ester structure together with an O atom to which the two pieces of R^{a} are linked; and X and Y are respectively protective groups which may be identical or may be different from each other.)

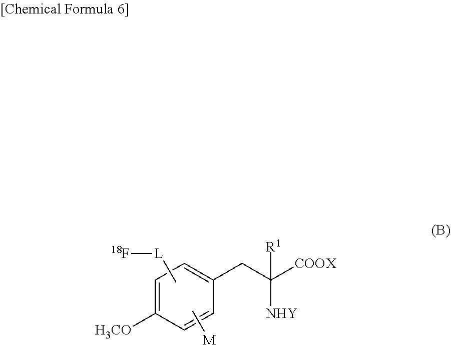

(in the formula, definitions of L, M, R¹, X, and Y are the same as the definitions in the formula (I), respectively.); and
the step ii) is a step of deprotecting protective groups X and Y in the compound represented by the formula (B) so as to obtain the compound represented by the formula (I);
<12> The production method according to <11>, in which the catalyst is a transition metal complex;
<13> The production method according to <11>, in which the catalyst is a copper complex;
<14> The method according to <11>, in which the deprotection in the step ii) includes treating the compound represented by the formula (B) under acidic conditions;
<15> The production method according to <11> to <14>, in which L is the direct bond; and M is the hydrogen atom; and
<16> The production method according to <11> to <15>, in which R¹ is the optionally substituted C₁ to C₅ alkyl group; R² is the hydrogen atom or the C₁ to C₅ alkyl group; and R³ is the hydrogen atom.

In a further aspect, the present invention also relates to a kit containing the intermediate compound to be used for synthesis of a compound represented by a formula (I), and provides
<17> A kit including the intermediate compound according to <9> or <10> and hydrogen fluoride (H¹⁸F), in which the intermediate compound and the hydrogen fluoride (H¹⁸F) are separately stored in the kit;
<18> The kit according to <17>, further including: a container storing a solution obtained by dissolving the intermediate compound in a solvent; and a container storing a solution obtained by dissolving the hydrogen fluoride (H¹⁸F) in a solvent;
<19> The kit according to <17> or <18>, being used for synthesis of the tracer composition for radioactive PET diagnosis according to <1>; and
<20> The kit according to <19>, in which the synthesis is performed by an automatic synthesis device.

### Advantageous Effects of Invention

According to the present invention, it is possible to provide an ¹⁸F-labeled tracer drug capable of reducing unfavorable renal uptake due to interaction with an organic anion transporter (OAT1) and having excellent accumulation properties and retention properties at a tumor site while maintaining high selectivity to the LAT1. Accordingly, it is possible to realize cancer diagnosis by radioactive PET with higher accuracy than before, and thus, for example, accurate evaluation of cancer metastasis can be performed, thereby making it possible to perform minimally invasive treatment without performing more than necessary resection, and to contribute to realization of appropriate and highly accurate medical care.

In addition, according to the production method of the present invention, the novel tracer compound can be efficiently synthesized in high yield by a method of label synthesis (¹⁸F⁻ method) using ¹⁸F-hydrogen fluoride (¹⁸F—HF). In particular, since synthesis using the ¹⁸F—HF as a raw material is a synthesis method similar to an existing tracer drug (¹⁸F-fluorodeoxyglucose or the like) widely used for cancer diagnosis as an insurance medical care in Japan at present, the synthesis can be easily introduced into many PET inspection facilities having a cyclotron, and thus it is useful for medical equipment of a synthesis device and practical application as a radiopharmaceutical.

### BRIEF DESCRIPTION OF DRAWINGS

FIG. 1 is a graph illustrating a measurement result of radiochemical purity of a tracer compound (¹⁸F-FAMT-OMe) of the present invention obtained in the Example.

FIG. 2 is a PET image taken over a certain period of time when the tracer compound (¹⁸F-FAMT-OMe) of the present invention was administered to a model mouse of a brain tumor (C6 glioma).

FIG. 3 is a graph illustrating changes over time in accumulation of the tracer compound of the present invention (¹⁸F-FAMT-OMe) and a comparative example compound (¹⁸F-FAMT) in the kidney.

FIG. 4 is a graph illustrating a SUV maximum value (SUVₘₐₓ) observed in the tracer compound of the present invention (¹⁸F-FAMT-OMe) and comparative examples (¹⁸F-FBPA and ¹⁸F-FAMT).

FIG. 5 is a graph illustrating a distribution (% ID/g) of the tracer compound of the present invention (¹⁸F-FAMT-OMe) in the mouse body.

### DESCRIPTION OF EMBODIMENTS

Hereinafter, embodiments of the present invention will be described. The scope of the present invention is not limited to these descriptions, and those other than examples to be described below may be appropriately modified and implemented without departing from the spirit of the present invention.

### 1. Definitions

In the present specification, terms “alkyl or alkyl group” may indicate any one of a linear group, a branched group, a cyclic group, or an aliphatic hydrocarbon group formed of a combination thereof. The number of carbon atoms in the alkyl group is not particularly limited, and is, for example, 1 to 20 carbon atoms (C_{1 to 20}), 1 to 15 carbon atoms (C_{1 to 15}), or 1 to 10 carbon atoms (C_{1 to 10}). In the present specification, the alkyl group may have one or more any substituents. For example, C_{1 to 8} alkyl includes methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. Examples of the substituent include an alkoxy group, a halogen atom (which may be any one of a fluorine atom, a chlorine atom, a bromine atom, and an iodine atom), an amino group, a mono- or di-substituted amino group, a substituted silyl group, acyl, and the like, but the examples thereof are not limited thereto. When the alkyl group has two or more substituents, the substituents may be identical or different. The same applies to alkyl moieties of other substituents including alkyl moieties (for example, an alkoxy group, an arylalkyl group, and the like).

In the present specification, “alkylene” is a divalent group formed of a linear or branched saturated hydrocarbon, and examples thereof include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, and 2,2-di-n-propyltrimethylene.

In the present specification, “aryl or aryl group” may be any one of a monocyclic aromatic hydrocarbon group and a fused polycyclic aromatic hydrocarbon group, and may contain one or more heteroatoms (for example, an oxygen atom, a nitrogen atom, a sulfur atom, and the like) as ring-constituting atoms. In this case, it may also be referred to as “heteroaryl” or “heteroaromatic”. Even in a case where aryl is any one of a monocyclic ring and a fused ring, aryl can be bonded at all possible positions. In the present specification, the aryl group may have one or more any substituents on its ring. Examples of the substituent can include an alkoxy group, a halogen atom, an amino group, a mono- or di-substituted amino group, a substituted silyl group, acyl, and the like, but the examples thereof are not limited thereto. When the aryl group has two or more substituents, the substituents may be identical or different. The same applies to aryl moieties of other substituents including aryl moieties (for example, an aryloxy group, an arylalkyl group, and the like).

In this specification, “arylalkyl” represents an alkyl substituted with the aryl. The arylalkyl may have one or more optional substituents. Examples of the substituent can include an alkoxy group, a halogen atom, an amino group, a mono- or di-substituted amino group, a substituted silyl group, an acyl group, and the like, but the examples thereof are not limited thereto. When the acyl group has two or more substituents, the substituents may be identical or different. Typical examples thereof include arylalkylbenzyl, p-methoxybenzyl, and the like.

In the present specification, the “halogen atom” can include a fluorine atom, a chlorine atom, a bromine atom, or an iodine atom.

In the present specification, when a certain functional group is defined as “optionally substituted”, a substituent type, a substitution position, and the number of substituents are not particularly limited, and when two or more substituents are included therein, the substituents may be identical or different. Examples of the substituent can include an alkyl group, an alkoxy group, a hydroxyl group, a carboxyl group, a halogen atom, a sulfo group, an amino group, an alkoxycarbonyl group, an oxo group, and the like, but the examples thereof are not limited thereto. Substituents may be further present in these substituents. For example, a halogenated alkyl group or the like can be cited, but the present invention is not limited thereto.

“Amide” used in the present specification includes both RNR′ CO— (in the case of R=alkyl, alkylaminocarbonyl-) and RCONR′— (in the case of R=alkyl, alkylcarbonylamino-).

“Ester” used in the present specification includes both ROCO— (in the case of R=alkyl, alkoxycarbonyl-) and RCOO— (in the case of R=alkyl, alkylcarbonyloxy-).

In the present specification, a specific substituent can form a ring structure with another substituent, and when such substituents are bonded to each other, those skilled in the art can understand that a specific substitution such as a bond to hydrogen is formed. Therefore, when a specific substituent is described as forming a ring structure together, those skilled in the art can understand that the ring structure can be formed by a normal chemical reaction and can be easily generated. All such ring structures and the formation processes thereof are within a recognition range of those skilled in the art. Further, the heterocyclic structure may have any substituent on the ring.

In the present specification, the term “ring structure” means a heterocycle ring or a carbocycle ring when the ring structure is formed by a combination of two substituents, and such ring can be saturated, unsaturated, or aromatic. Thus, cycloalkyl, cycloalkenyl, aryl, and heteroaryl defined above are included.

### 2. Tracer Composition

A composition of the present invention is a tracer composition for radioactive PET diagnosis that targets an L-type amino acid transporter 1 (LAT1), and the tracer composition is characterized by containing an amino acid derivative compound represented by the following formula (I) or a pharmaceutically acceptable salt thereof.

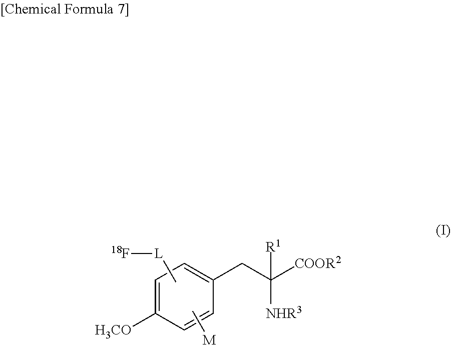

The compound of the formula (I) is a compound having a structure in which a methoxy (OMe) group is introduced at the 4-position of a benzene ring of an α-methyltyrosine (AMT) skeleton, which is an amino acid derivative that has affinity for the LAT1, and a radioactive fluorine atom (¹⁸F), which is a PET labeling element, is introduced as a substituent. By having such a structure, the compound has a property of reducing unfavorable renal uptake due to interaction with an organic anion transporter (OAT1), thereby having excellent accumulation properties and retention properties at a tumor site while maintaining high selectivity to the LAT1.

Here, the LAT1 is a type of amino acid transporter, which is a membrane protein necessary for intracellular uptake of neutral branched chain amino acids and aromatic amino acids. Further, the LAT1 has been shown to be specifically expressed in cancer and is responsible for supplying amino acids as nutrients in cancer tissues. The term “having affinity for the LAT1” means being selectively incorporated into the LAT1. More preferably, it means having a property of being more easily incorporated into the LAT1 than an amino acid transporter (for example, LAT2) in normal cells.

In the formula (I), L is direct bond or optionally substituted a C₁ to C₅ alkylene group; M is a hydrogen atom or a halogen atom; R¹ is a hydrogen atom or an optionally substituted C₁ to C₅ alkyl group; R² is a hydrogen atom, a C₁ to C₅ alkyl group, a C₆ to C₁₄ aryl group, or a C₇ to C₁₆ arylalkyl group; and R³ is a hydrogen atom or a C₁ to C₃ alkyl group. In the present specification, for example, C₁ to C₅ represent that the number of carbon atoms is 1 to 5.

Preferably, L is a direct bond; and M is a hydrogen atom. Similarly, preferably R¹ is the optionally substituted C₁ to C₅ alkyl group; R² is the hydrogen atom or the C₁ to C₅ alkyl group; and R³ is the hydrogen atom.

¹⁸F-L can be present at any position on the benzene ring, but is preferably present at the ortho position with an OCH₃ group. More preferably, ¹⁸F-L can be at the ortho position with the OCH₃ group on the benzene ring and can be at the meta position with a side chain (a linking group to a moiety having R¹, COOR², and NHR³).

As a non-limiting specific example of the compound represented by the formula (1), the following compound in which L is a direct bond and M is a hydrogen atom can be cited. This compound is obtained by directly introducing ¹⁸F onto the benzene ring of α-methyltyrosine (in the present specification, also referred to as “¹⁸F-FAMT-OMe”).

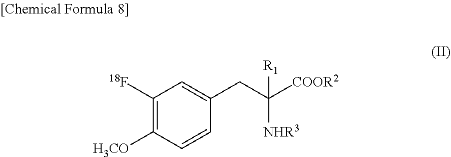

In formula (II), definitions of R¹, R², and R³ are the same as those of the formula (I), respectively.

The compounds represented by the formula (I) of the present invention can be in the form of pharmaceutically acceptable salts thereof. Such salts are not particularly limited, and examples thereof can include a base addition salt, an acid addition salt, an amino acid salt, and the like. Examples of the base addition salt can include alkali metal salts such as a sodium salt, a potassium salt, and a lithium salt; alkaline earth metal salts such as a calcium salt and a magnesium salt; ammonium salts such as a tetramethylammonium salt and a tetrabutylammonium salt; and organic amine salts such as a triethylamine salt, a methylamine salt, a dimethylamine salt, a cyclopentylamine salt, a benzylamine salt, a phenethylamine salt, a piperidine salt, a monoethanolamine salt, a diethanolamine salt, a tris(hydroxymethyl)methylamine salt, a lysine salt, an arginine salt, a N-methyl-D-glucamine salt, and a morpholine salt. Examples of the acid addition salt can include mineral acid salts such as hydrochloride, hydrobromide, sulfate, nitrate, and phosphate; and organic acid salts such as methanesulfonic acid, benzenesulfonic acid, para-toluenesulfonic acid, acetic acid, propionate, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, and salicylic acid. Examples of the amino acid salt include a glycine salt, an aspartic acid salt, and a glutamic acid salt. In addition, a metal salt such as an aluminum salt may be used.

The compound represented by the formula (I) of the present invention can be a D-form or an L-form and is preferably the L-form. In addition, the compound represented by the formula (I) may have one or two or more asymmetric carbons depending on the type of substituent, and stereoisomers such as optical isomers or diastereoisomers may exist. Stereoisomers in pure form, any mixture of the stereoisomers, racemates and the like are all encompassed within the scope of the present invention.

The compound represented by the formula (I) of the present invention or a pharmaceutically acceptable salt thereof may exist as a hydrate or a solvate, and all of these materials are included within the scope of the present invention. The type of solvent that forms the solvate is not particularly limited, and examples thereof can include solvents such as ethanol, acetone, and isopropanol.

As described above, the tracer composition for radioactive PET diagnosis of the present invention is a composition containing the compound represented by the formula (I) or the pharmaceutically acceptable salt thereof. Here, the term “composition” includes not only a product containing an active ingredient and an inactive ingredient (pharmaceutically acceptable excipient) that constitutes a carrier, but also any products produced directly or indirectly as a result of association, complexation or aggregation of any two or more ingredients, as a result of dissociation of one or more ingredients, or as a result of another type of reaction or interaction of one or more ingredients.

The compound represented by the formula (I) in the tracer composition for radioactive PET diagnosis of the present invention functions as an imaging probe compound capable of detecting cancer tissue by its affinity for the LAT1, that is, by being selectively taken up and accumulated in cancer cells in which the LAT1 is expressed. Therefore, the tracer composition for radioactive PET diagnosis of the present invention is preferably used for diagnosing cancer. Cancer targeted by the tracer composition of the present invention is not particularly limited as long as the cancer is a cancer cell in which the LAT1 is expressed, and includes any malignant tumor. Examples of cancer can include pancreatic cancer, large bowel cancer, lung cancer, prostate cancer, stomach cancer, breast cancer, kidney cancer, laryngeal cancer, esophageal cancer, liver cancer, and brain tumor. Preferably, the tracer composition of the present invention can be used for intractable cancer and advanced cancer that is difficult to be treated with a surgical operation.

It is noted that affinity for the LAT1 can be determined, for example, by an amino acid uptake inhibition test using a human LAT1 stable expression cell line and a human LAT2 stable expression cell line (Khunweeraphong et al J Pharmacol Sci. 2012 Aug. 18; 119(4):368-80. Epub 2012 Jul. 31.). A cell having a higher amino acid uptake inhibition rate in the human LAT1 stable expression cell line than an uptake inhibition rate in the human LAT2 stable expression cell line can be selected as a compound having cancer cell-specific accumulation activity.

The tracer composition for radioactive PET diagnosis of the present invention can be formulated by appropriately blending a pharmaceutically acceptable carrier and an additive in addition to the compound represented by the formula (I). The tracer composition is preferably a liquid preparation, particularly preferably an injection. Administration to a subject may be local administration or systemic administration, and the systemic administration is preferable. An administration route is not particularly limited, but intravenous injection or drip infusion is preferable. Preparation of injections can be performed by a method known to the public in the field concerned. The solution may be prepared, for example, by dissolving the compound represented by the formula (I) in an appropriate liquid carrier (water for injection, physiological saline, Ringer's solution, or the like), sterilizing the liquid carrier using a filter or the like, and then filling the sterilized liquid carrier into an appropriate container such as a vial or an ampoule. When the compound is dissolved, a suitable dissolution auxiliary agent such as alcohol, polyalcohol, nonionic surfactant, or the like may be used. Furthermore, sugar or a sugar alcohol may be added as an additive, preferably the sugar alcohol is used, and examples of the sugar alcohol include erythritol, xylitol, sorbitol, mannitol, and the like. Further, the solution can be lyophilized and restored to solution again with an appropriate liquid carrier when used. A suspension may be prepared, for example, by sterilizing the compound represented by the formula (I) using, for example, ethylene oxide, and then suspending the sterilized compound in a sterilized liquid carrier.

Those skilled in the art can appropriately select a type of an additive for preparation used in the production of the tracer composition of the present invention, a ratio of the additive for preparation to an active ingredient, or a production method of the pharmaceutical composition, depending on the form of the composition. An inorganic or organic substance or a solid or liquid substance can be used as the additive for preparation, and generally, the additive can be blended between 1% to 90% by weight relative to a component weight of the compound represented by the formula (I).

As procedures of performing PET measurement using the tracer composition for radioactive PET diagnosis of the present invention, typically, the tracer composition of the present invention is administered to a subject as an injection, measurement is performed using a known positron emission tomography device, and an internal distribution and a degree of accumulation of the compound represented by the formula (I) are measured. In cancer tissue detection, a tissue having a relatively large radiation dose can be detected as a cancer-causing tissue by comparing the radiation dose of each tissue. In comparison therebetween, it is preferable to use a standardized uptake value (SUV) or an SUV (tissue)/SUV (blood), which is a relative value based on the radiation dose of blood. Alternatively, a tissue having a relatively large radiation dose may be identified from an image.

In the tracer composition for radioactive PET diagnosis of the present invention, an amount of radioactivity when the tracer composition is administered to humans is not particularly limited as long as the amount of radioactivity necessary for evaluation can be secured at the time of use, but the amount thereof is preferably 74 to 370 MBq at the time of use and is more preferably adjusted depending on body weight and the like.

In addition, since the tracer composition for radioactive PET diagnosis of the present invention is considered to be accumulated a lot in cancer cells having a high proliferation rate, the tracer composition for radioactive PET diagnosis can be used to evaluate a degree of malignancy of cancer. The degree of malignancy of cancer can be quantitatively evaluated by visually evaluating or analyzing the radiation dose or the image of cancer tissue. It can be determined that as the radiation dose in cancer tissue is higher, the degree of malignancy is higher (faster proliferation rate). An evaluation result of the degree of malignancy of cancer can be used to confirm treatment effects, to determine treatment policies, and the like.

In a preferred aspect, the tracer composition of the present invention can further contain a pharmaceutical compound for cancer treatment. The compound of the formula (I) capable of detecting cancer tissue is used in combination with the pharmaceutical compound for cancer treatment, thereby making it possible to monitor arrival of a drug to an affected area, a state of the affected area after administration, and the like at the same time as treatment. Such a pharmaceutical compound is preferably a compound having therapeutic ability with an α-ray beam or a β-ray beam, and for example, the pharmaceutical compound can be a compound containing astatine-211 (²¹¹At) in a molecule and having α-ray therapeutic ability.

### 3. Production Method of Tracer Compound and Intermediate Compound

In another aspect, the present invention also relates to a production method of the tracer compound represented by the formula (I). The production method uses a novel intermediate compound (a compound of formula (A) below) having a structure in which a boronic acid ester leaving group such as a pinacol ester is introduced into the 3-position of a benzene ring of an α-methyltyrosine (AMT) skeleton, thereby making it possible to efficiently synthesize the tracer compound of the formula (I) with high yield by a method of label synthesis using ¹⁸F-hydrogen fluoride (¹⁸F—HF) (¹⁸F⁻ method) instead of conventional label synthesis using ¹⁸F—F₂ gas (¹⁸F⁺ method). Accordingly, the present invention also relates to such a novel intermediate compound of the formula (A).

More specifically, the production method of the present invention is characterized by including the following steps i) and ii).

### Step i)

A step of adding hydrogen fluoride (H¹⁸F) to a compound represented by the formula (A) in the presence of a catalyst to obtain a compound represented by the following formula (B).

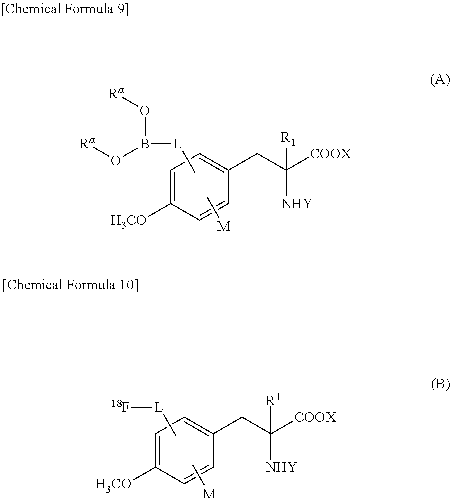

### Step ii)

A step of obtaining the compound represented by the formula (I) by deprotecting protective groups X and Y in the compound represented by the formula (B).

The step i) is a step of introducing a radioactive fluorine atom (¹⁸F) which is a PET labeling element, by label synthesis using ¹⁸F-hydrogen fluoride (¹⁸F—HF), into a novel intermediate compound of the formula (A) having a pinacol ester as a leaving group at the 3-position of the benzene ring of the α-methyltyrosine (AMT) skeleton.

In the formula (A), definitions of L, M, and R¹ are the same as those of the formula (I), respectively. Respective pieces of R^{a} may be identical or different and each R^{a} independently represents a hydrogen atom or an optionally substituted C₁ to C₅ alkyl group, and when all R^{a}s are C₁ to C₅ alkyl groups, two pieces of R^{a} may form a cyclic ester structure together with an O atom to which the two pieces of R^{a} are linked; and X and Y are protective groups that may be identical or different. Preferably, L is a direct bond; and M is a hydrogen atom. Similarly, preferably R¹ is the optionally substituted C₁ to C₅ alkyl group; R² is the hydrogen atom or the C₁ to C₅ alkyl group; and R³ is the hydrogen atom.

Here, the term “protective group” refers to a group that prevents or inhibits undesirable chemical reactions and is designed to be sufficiently reactive so as to obtain a desired product by being detached from a functional group in question under mild conditions to such an extent that the rest of the molecule is not altered.

The protective group in X of the formula (A) is not particularly limited as long as the protective group is generally used as a protective group of a carboxyl groups, and examples thereof include a methyl group, an ethyl group, a propyl group, an isopropyl group, a butyl group, an isobutyl group, a sec-butyl group, a tert-butyl group, a hexyl group, a bromo-tert-butyl group, a trichloroethyl group, a benzyl group, a p-nitrobenzyl group, an o-nitrobenzyl group, a p-methoxybenzyl group, a diphenylmethyl group, a trityl group, a p-tert-butylbenzyl group, an acetoxymethyl group, a propionyloxymethyl group, a butyryloxymethyl group, an isobutyryloxymethyl group, a valeryloxymethyl group, a pivaloyloxymethyl group, an acetoxyethyl group, an acetoxypropyl group, an acetoxybutyl group, a propionyloxyethyl group, a propionyloxypropyl group, a butyryloxyethyl group, an isobutyryloxyethyl group, a pivaloyloxyethyl group, a hexanoyloxyethyl group, an ethylbutyryloxymethyl group, a dimethylbutyryloxymethyl group, a pentanoyloxyethyl group, a methoxycarbonyloxymethyl group, an ethoxycarbonyloxymethyl group, a propoxycarbonyloxymethyl group, a tert-butoxycarbonyloxymethyl group, a methoxycarbonyloxyethyl group, an ethoxycarbonyloxyethyl group, an isopropoxycarbonyloxyethyl group, a tert-butyldimethylsilyl group, a trimethylsilyl group, a methoxymethyl group, an ethoxymethyl group, a propoxymethyl group, an isopropoxymethyl group, a (2-methylthio)-ethyl group, a 3-methyl-2-butenyl group, a 5-indanyl group, and a 3-phthalidyl group. The tert-butyl group, the benzyl group, the p-methoxybenzyl group, the diphenylmethyl group, and the trityl group are preferably used.

A protective group in Y of the formula (A) is not particularly limited as long as the protective group is generally used as a protective group for an amino group, and examples thereof includes a formyl group, a phenylcarbonyl group, a methoxycarbonyl group, an ethoxycarbonyl group, a tert-butoxycarbonyl (Boc) group, a phenyloxycarbonyl group, a 9-fluorenylmethyloxycarbonyl group, an adamantyloxycarbonyl group, a benzyloxycarbonyl group, a benzyl carbonyl group, a benzyl group, a benzhydryl group, a trityl group, and a phthaloyl group. The tert-butoxycarbonyl (Boc) group, the trityl group, and the benzyloxycarbonyl group are preferably used.

As the cyclic ester structure formed by the two pieces of R^{a} in the formula (A), 5 to 10 membered cyclic esters can be used, but the pinacol ester is preferably used. A specific example of the compound of the formula (A) having the pinacol ester is a compound represented by formula (A′) shown below.

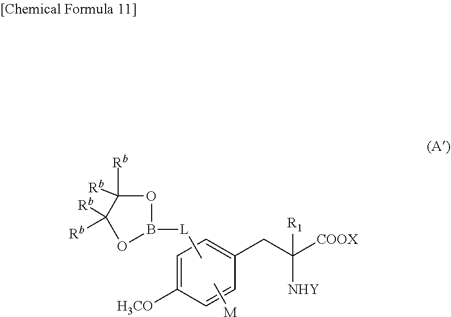

(In the formula, definitions of L, M, R¹, X, and Y are the same as those of the formula (A), respectively; and the respective pieces of R^{b} may be identical or different and each R^{b} independently represents a hydrogen atom or an optionally substituted C₁ to C₅ alkyl group.)

The catalyst used in step i) is preferably a transition metal complex, and can be, for example, a transition metal complex having pyridine or triflate as a ligand. The transition metal is preferably copper, palladium, or nickel.

Radioactive fluorine (¹⁸F) in the hydrogen fluoride (¹⁸F—HF) used in the step i) can be obtained by a known method such as a method of performing proton irradiation using H₂¹⁸O concentrated water as a target. Various methods can be used. For example, the H₂¹⁸O concentrated water containing the radioactive fluorine is caused to pass through, for example, an anion exchange column, and the radioactive fluorine is adsorbed and collected by the column to separate the radioactive fluorine from the H₂¹⁸O concentrated water. Thereafter, potassium carbonate solution is eluted, an interphase transfer catalyst is added, and a mixture thereof is dried and used. Alternatively, the radioactive fluorine is eluted with a tetra-N-butylammonium hydrogen carbonate solution to react the solution as it is.

The step i) can be typically performed at a temperature condition of 80 to 150° C.

Next, the step ii) is a step of deprotecting the protective groups X and Y in the compound of the formula (B) obtained in the step i) and obtaining the target tracer compound represented by the formula (I).

As a method of deprotection of X and Y, reaction conditions, and the like, those known in the relevant technical field can be used, and typically, the compound represented by the formula (B) can be treated under acidic conditions. For example, hydrochloric acid (HCl) can be added to make a reaction solution acidic conditions.

The step ii) can be typically performed at a temperature condition of 50 to 110° C.

If necessary, a step of purifying the compound of the formula (I), which is a product, may be performed after the step ii). Such purification can be preferably performed by column chromatography or HPLC. For example, in addition to reverse phase chromatography, cation exchange column chromatography may be performed. Thereafter, the desired product may be separated and purified by performing anion exchange column chromatography. It is noted that, in a stationary phase such as an ion exchange resin, those commonly used in the technical field can be used.

Although reaction conditions such as a solvent and a reaction temperature in each step in the production method of the present invention described above will be described in detail as representative examples in the following embodiments, the present invention is not necessarily limited thereto, and those skilled in the art can appropriately select each reaction condition based on general knowledge in organic synthesis. In particular, as described below, the production method of the present invention can be performed using an automatic synthesis device commonly used in PET facilities.

### 4. Kit

In another aspect, the present invention also relates to a kit in which the intermediate compound represented by the formula (A) and the hydrogen fluoride (H¹⁸F) are separately stored. The kit is suitable for the synthesis of a tracer composition for radioactive PET diagnosis containing the compound of the formula (I) of the present invention, and is particularly suitable for ¹⁸F-label synthesis using an automatic synthesis device commonly used in PET facilities. Such an automatic synthesis device is widely commercially available as a device capable of automatically synthesizing medical compounds containing radioactive elements.

Typically, the kit of the present invention is configured to include a container storing a solution obtained by dissolving the intermediate compound of the formula (A) in a solvent and a container storing a solution obtained by dissolving hydrogen fluoride (H¹⁸F) in a solvent.

The concentration and solution amount of the intermediate compound and the hydrogen fluoride in the solution stored in each container of the kit can be appropriately set depending on the required amount of the product (compound of the formula (I)).

Generally, in the automatic synthesis device, a kit that stores such raw material compounds is sometimes referred to as a “cassette”. The cassette is a disposable member containing a reagent, a reaction container, and equipment necessary to carry out production of a radioactive fluorinated containing compound and is designed to be detachably and interchangeably attached to the automatic synthesis device. The cassette has a plurality of reaction containers having a volume of 0.5 to 10 mL, preferably 0.5 to 5 mL, most preferably 0.5 to 4 mL, and a reagent or a solvent can be stored within the reaction container. It is advantageous in that various radioactive fluorinated containing compounds can be automatically produced while minimizing the risk of radioactive contamination only by replacing the cassette.

For example, the cassette for the automatic synthesis device includes a plurality of linearly arranged rows of valves, and each of the valves has a structure coupled to a port on which a reagent or a vial can be mounted by a needle puncture of an inverted septum sealed vial or by an airtight coupling joint thereof. Preferably, the cassette includes 15 to 40 valves and most preferably 20 to 30 valves. Here, the valve has a fitting type joint that is engaged with a corresponding movable arm of the automatic synthesis device, and when the cassette is mounted on the automatic synthesis device, external rotation of the arm controls opening and closing of the valve. The additional movable member of the automatic synthesis device is designed to grip a plunger tip of a syringe and to raise or lower a syringe outer cylinder.

Since the production method of the present invention using a method of label synthesis (¹⁸F⁻ method) by the ¹⁸F-hydrogen fluoride (¹⁸F—HF) is a synthesis method similar to an existing tracer drug (¹⁸F-fluorodeoxyglucose or the like) widely used for cancer diagnosis, using the above-described kit provides an advantageous effect in that it is possible to easily perform a test in PET testing facilities having the automatic synthesis device.

### EXAMPLES

Hereinafter, the present invention will be described in more detail with reference to Examples, but the present invention is not limited thereto.

### Example 1

### 1. Synthesis of Tracer Compound Labeled with ¹⁸F: Synthesis of α-Methyltyrosine Derivative (¹⁸F-FAMT-OMe)

A compound 3 (¹⁸F-FAMT-OMe), which is a tracer compound of the formula (I), was synthesized by a synthesis scheme represented below.

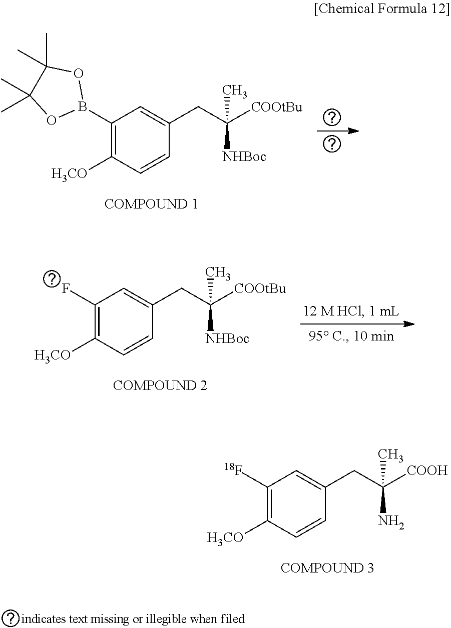

### (1) Synthesis of Compound 1

An intermediate compound (compound 1) having a pinacol ester was synthesized by the following procedures. 2-Bromo-4-(bromomethyl)-1-methoxybenzene (31.4 mmol), 2-[(4-chlorobenzyliden)amino]propanoic acid tert-butyl ester (24.9 mmol), and 4,4-dibutyl-2,6-bis(3,4,5-trifluorophenyl)-4,5-dihydro-3H-dinaphtho[2,1-c:1′,2′-e]azepinium bromide (0.026 mmol) were dissolved in 53 mL of toluene. Thereafter, under stirring, an 80% aqueous cesium hydroxide solution was added at −5° C., and a mixture thereof was stirred at 0° C. for 18 hours. After 50 mL of water was added to a reaction solution and was diluted, an organic phase was separated. After performing a drying procedure using anhydrous MgSO₄, the solvent was distilled under reduced pressure. The 100 mL of THF was added to the product and was dissolved. A solution obtained by dissolving citric acid monohydrate (199.2 mmol) in 140 mL of water was added to this solution, and a mixture thereof was stirred at room temperature for 3 hours. After the reaction, THF was distilled off under reduced pressure, and the aqueous phase was washed with 50 mL of diethyl ether, and then K₂CO₃ was added to make it basic. This solution was extracted with ethyl acetate and was dried with anhydrous MgSO₄, and then the solvent was distilled off under reduced pressure. The product was purified by silica gel chromatography. Boc₂O (22.1 mmol) and Na₂CO₃ (30.2 mmol) were dissolved in the obtained compound (20.1 mmol) in 57 mL of acetonitrile, and a mixture thereof was stirred at room temperature for 18 hours. The reaction solution was filtered, then the solvent was distilled off under reduced pressure, and the product was subjected to the silica gel chromatography for purification. The obtained compound (5.18 mmol) was dissolved in 23 mL of DMSO, and Bis (pinacolato) diborane (10.4 mmol), Pd (dba)₂ (0.259 mmol), XPhos (0.518 mmol), and potassium acetate (15.5 mmol) were added, and then a mixture thereof was stirred at 80° C. for 5 hours. The reaction solution was cooled, then 46 mL of water was added thereto, and a mixture thereof was extracted with 100 mL of ethyl acetate. The extracted organic phase was washed with saturated saline and then was dried over anhydrous MgSO₄. After the solvent was distilled under reduced pressure, a product is purified by silica gel chromatography and the compound 1 is obtained.

### (2) Synthesis of Compound 2

Subsequently, the obtained compound 1 was labeled with ¹⁸F to synthesize a compound 2. Specifically, first, ¹⁸F—HF produced by a cyclotron was concentrated and purified by an anion exchange column (QMA), and then collected in a reaction container having 0.5 mL of a TEAHCO₃/methanol solution. After the solvent was distilled off, 1 mL of dimethylacetamide obtained by dissolving the compound 1 (20 μmol) and Cu(OTf)₂(Py)₄ (60 μmol) was added, and fluorination was performed at 110° C. for 20 minutes with stirring, thereby obtaining the compound 2.

### (3) Synthesis of Compound 3

Subsequently, the protective group of the obtained compound 2 was deprotected to synthesize a compound 3 (¹⁸F-FAMT-OMe), which is a target tracer compound. Specifically, the reaction solution containing the obtained compound 2 was diluted with ethanol, the whole amount of the diluted reaction solution was passed through a column using silica as a carrier, and then the passed liquid was collected in another reaction container. After the solvent was distilled off, 1 mL of 12 M hydrochloric acid was added, and deprotection was performed at 95° C. for 10 minutes. After 1 mL of injection water was added to this reaction solution and was diluted, an entire amount thereof was injected into HPLC, and separation and purification was performed using a reverse phase column, thereby obtaining the compound 3.

### (4) Measurement of Radiochemical Purity

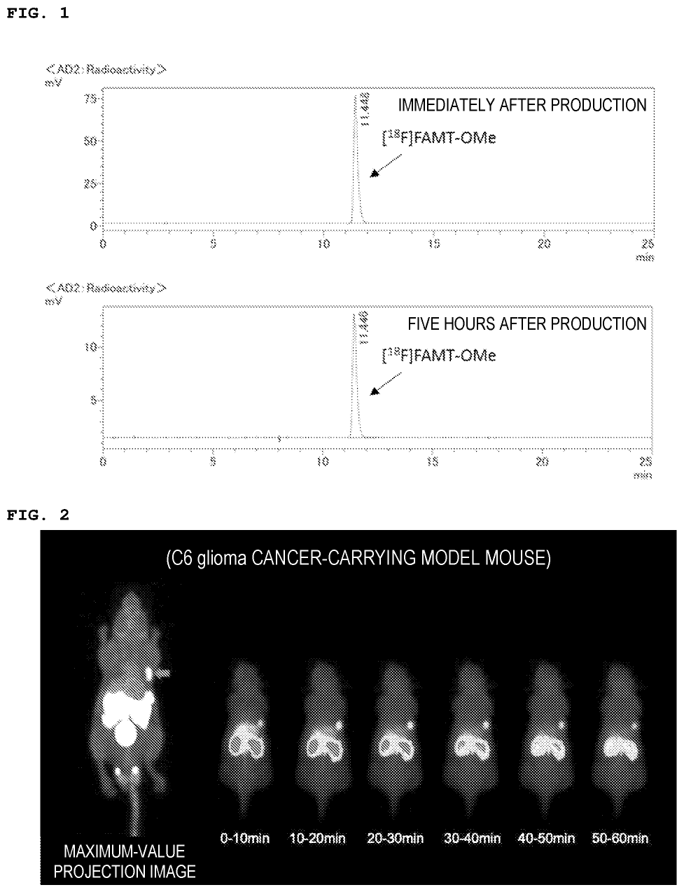

A result of measuring radiochemical purity of the obtained compound 3 is illustrated in FIG. 1. As a result, it was confirmed that the compound 3 had a radiochemical purity of 99.9% or more immediately after producing the same, and the purity was maintained even after 5 hours of production.

### Example 2

### 3. Evaluation of Accumulation Properties of ¹⁸F-FAMT-OMe in Cancer Cells

¹⁸F-FAMT-OMe synthesized in the Example 1 was administered to a model mouse of brain tumor (C6 glioma), and accumulation properties thereof were observed Specifically, a solution containing about 10 MBq of ¹⁸F-FAMT-OMe was intravenously injected into the mouse, and PET images were acquired at regular intervals. As a result, as illustrated in FIG. 2, it was recognized that ¹⁸F-FAMT-OMe of the present invention had clear and high accumulation at a tumor site, and washing out was significantly gentle in comparison with the conventional ¹⁸F-FAMT that does not have an OH methoxy group. In particular, in the ¹⁸F-FAMT-OMe of the present invention, the accumulation one hour after administration was better than that of the conventional ¹⁸F-FAMT.

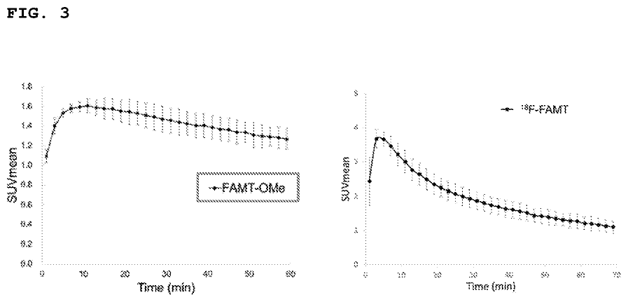

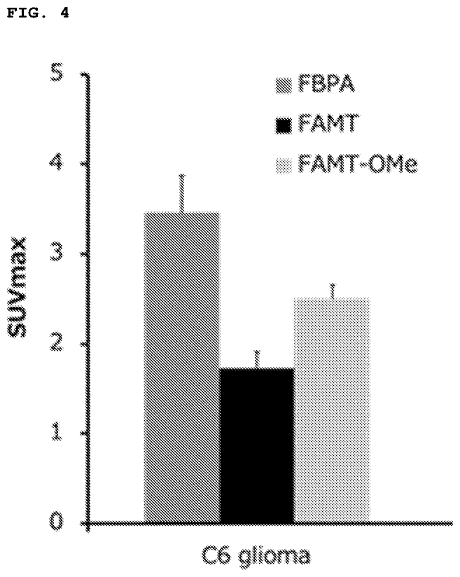

In addition, FIG. 3 (left drawing) illustrates a result of measuring changes over time in the accumulation of the ¹⁸F-FAMT-OMe in the kidney on the basis of a standardized uptake value (SUV) value based on the radiation dose of blood. As a comparative example, a result of a case using the ¹⁸F-FAMT is illustrated in FIG. 3 (right drawing). In addition, FIG. 4 is a graph comparing the maximum value of SUV (SUVₘₐₓ) with the existing tracer compounds ¹⁸F-FBPA and ¹⁸F-FAMT.

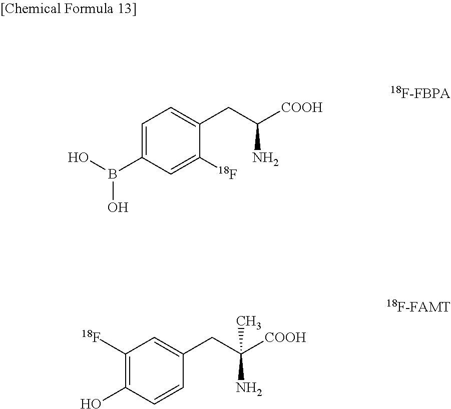

As a result, since the ¹⁸F-FAMT-OMe of the present invention was excreted from the kidney over time, it was demonstrated that the ¹⁸F-FAMT-OMe was superior to the ¹⁸F-FAMT of the comparative example as a LAT1-specific tracer compound for PET.

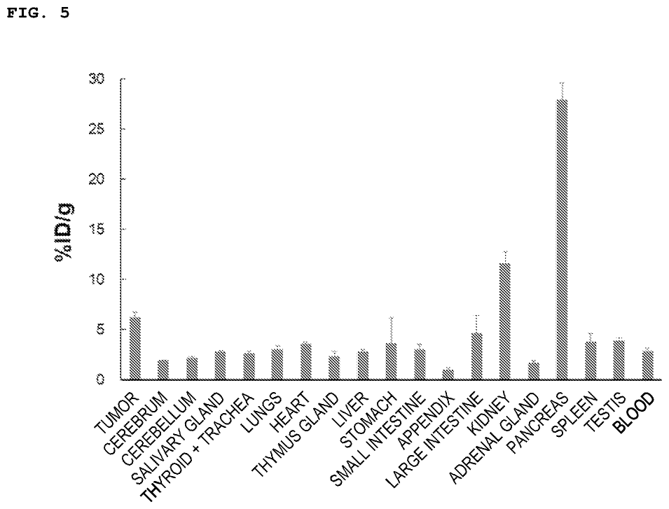

Further, FIG. 5 is a graph representing a distribution (% ID/g) of the ¹⁸F-FAMT-OMe of the present invention in the mouse body. As a result, in the kidney, which is an excretory organ, and in a mouse, except for the pancreas in which LAT1 expression was recognized, significantly higher accumulation at a tumor site was confirmed as compared with accumulation in normal organs and blood.

As described above, according to the tracer compound of the present invention, in cases where it is difficult to distinguish between inflammatory accumulation and lymph node metastasis using conventional tracer compounds (FDG and the like) (particularly, patients before surgery), it becomes possible to perform accurate metastasis evaluation.

1. A tracer composition for radioactive PET diagnosis, the composition targeting an L-type amino acid transporter 1 (LAT1) and comprising a compound represented by a following formula (I) or a pharmaceutically acceptable salt thereof: 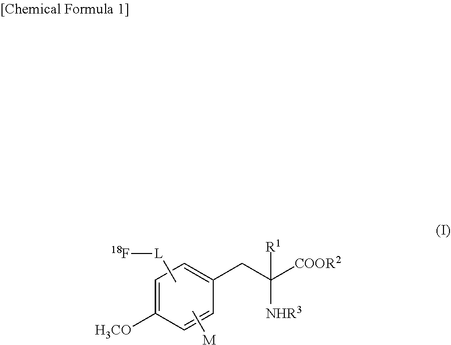
(in the formula, L is a direct bond or an optionally substituted C₁ to C₅ alkylene group; M is a hydrogen atom or a halogen atom; R¹ is the hydrogen atom or an optionally substituted C₁ to C₅ alkyl group; R² is the hydrogen atom, the C₁ to C₅ alkyl group, a C₆ to C₁₄ aryl group, or a C₇ to C₁₆ arylalkyl group; and R³ is the hydrogen atom or a C₁ to C₃ alkyl group.).

2. The composition according to claim 1, wherein L is the direct bond; and M is the hydrogen atom.

3. The composition according to claim 1, wherein the compound is represented by a following formula (II): 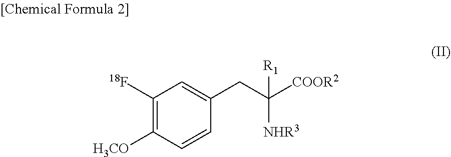
(in the formula, definitions of R¹, R², and R³ are the same as definitions of the formula (I), respectively).

4. The composition according to any one of claims 1 to 3, wherein R¹ is the optionally substituted C₁ to C₅ alkyl group; R² is the hydrogen atom or the C₁ to C₅ alkyl group; and R³ is the hydrogen atom.

5. The composition according to any one of claims 1 to 4, being used for a diagnosis of cancer.

6. The composition according to any one of claims 1 to 5, further comprising a pharmaceutical compound for cancer treatment.

7. The composition according to any one of claims 1 to 5, being used in combination with a pharmaceutical compound for cancer treatment.

8. The tracer composition according to claim 6 or 7, wherein the pharmaceutical compound for cancer treatment is a compound containing astatin-211 (²¹¹At) in a molecule.

9. An intermediate compound represented by a following formula (A) or a pharmaceutically acceptable salt thereof: 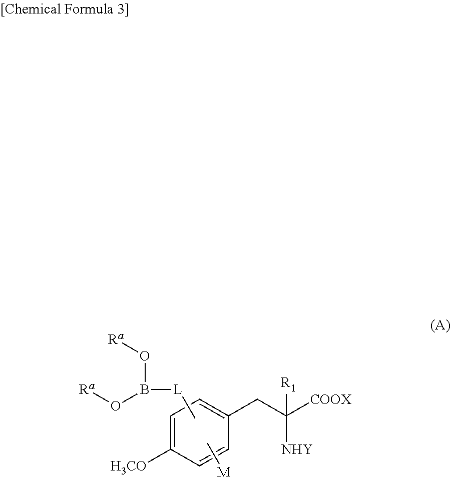
(in the formula, L is a direct bond or an optionally substituted C₁ to C₅ alkylene group; M is a hydrogen atom or a halogen atom; respective pieces of R^{a} may be identical or may be different from each other, wherein each of the pieces of R^{a} independently represents the hydrogen atom or an optionally substituted C₁ to C₅ alkyl group, and wherein, when all the pieces of R^{a} are the C₁ to C₅ alkyl group, the two pieces of R^{a} may form a cyclic ester structure together with an O atom to which the two pieces of R^{a} are linked; R¹ is the hydrogen atom or the optionally substituted C₁ to C₅ alkyl group; and X and Y are respectively protective groups which may be identical or may be different from each other.).

10. The intermediate compound according to claim 9, wherein X is a group selected from a group formed of a tert-butyl group, a benzyl group, an acetyl group, a benzoyl group, and an alkyl group; and Y is a group selected from a group formed of a tert-butoxycarbonyl group, a benzyloxycarbonyl group, a 9-fluorenylmethyloxycarbonyl group, an allyloxycarbonyl group, a 2,2,2-trichloroethoxycarbonyl group, and a 2-(trimethylsilyl)ethoxycarbonyl group.

11. A production method of a compound represented by a following formula (I), 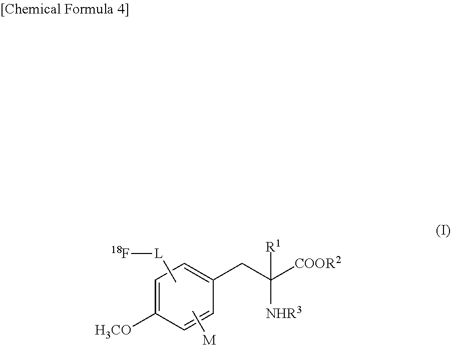
(in the formula, L is a direct bond or an optionally substituted C₁ to C₅ alkylene group; M is a hydrogen atom or a halogen atom; R¹ is the hydrogen atom or an optionally substituted C₁ to C₅ alkyl group; R² is the hydrogen atom, the C₁ to C₅ alkyl group, a C₆ to C₁₄ aryl group, or a C₇ to C₁₆ arylalkyl group; and R³ is the hydrogen atom or a C₁ to C₃ alkyl group.), the production method comprising a step i) and a step ii),
wherein the step i) is a step of adding hydrogen fluoride (H¹⁸F) to a compound represented by a formula (A) in presence of a catalyst so as to obtain a compound represented by a following formula (B) 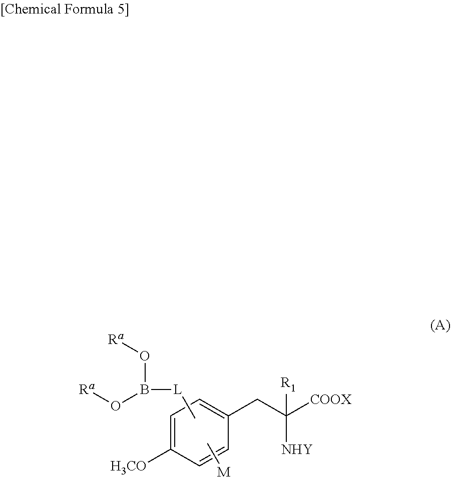
(in the formula, definitions of L, M, and R¹ are the same as definitions of the formula (I), respectively; respective pieces of R^{a} may be identical or different from each other, wherein each of the pieces of R^{a} independently represents the hydrogen atom or the optionally substituted C₁ to C₅ alkyl group, and wherein, when all the pieces of R^{a} are the C₁ to C₅ alkyl group, the two pieces of R^{a} may form a cyclic ester structure together with an O atom to which the two pieces of R^{a} are linked; and X and Y are respectively protective groups which may be identical or may be different from each other.) 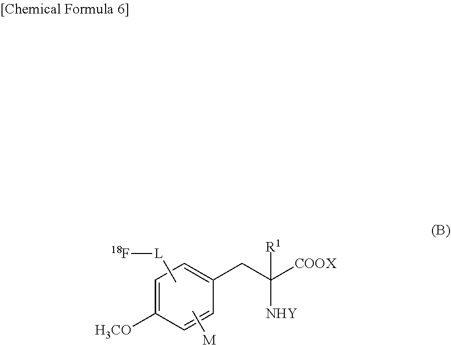
(in the formula, definitions of L, M, R¹, X, and Y are the same as the definitions in the formula (I), respectively.), and
wherein the step ii) is a step of deprotecting protective groups X and Y in the compound represented by the formula (B) so as to obtain the compound represented by the formula (I).

12. The production method according to claim 11, wherein the catalyst is a transition metal complex.

13. The production method according to claim 11, wherein the catalyst is a copper complex.

14. The method according to claim 11, wherein the deprotection in the step ii) includes treating the compound represented by the formula (B) under acidic conditions.

15. The production method according to claims 11 to 14, wherein L is the direct bond; and M is the hydrogen atom.

16. The production method according to claims 11 to 15, wherein R¹ is the optionally substituted C₁ to C₅ alkyl group; R² is the hydrogen atom or the C₁ to C₅ alkyl group; and R³ is the hydrogen atom.

17. A kit comprising the intermediate compound according to claim 9 or 10 and hydrogen fluoride (H¹⁸F), wherein the intermediate compound and the hydrogen fluoride (H¹⁸F) are separately stored in the kit.

18. The kit according to claim 17, further comprising: a container storing a solution obtained by dissolving the intermediate compound in a solvent; and a container storing a solution obtained by dissolving the hydrogen fluoride (H¹⁸F) in a solvent.

19. The kit according to claim 17 or 18, being used for synthesis of the tracer composition for radioactive PET diagnosis according to claim 1.

20. The kit according to claim 19, wherein the synthesis is performed by an automatic synthesis device.
